(19) United States
(12) Patent Application Publication
(10) Pub. No.: US 2019/0125211 A1
(43) Pub. Date: May 2, 2019

(54) PORTABLE DEVICES AND METHODS FOR DETECTING AND IDENTIFYING COMPOUNDS IN BREATH

(71) Applicant: NGageIT Digital Health, Inc., Toronto (CA)

(72) Inventors: Satya Reddy, Burlington (CA); Jennifer Murdoch, Burlington (CA); Raj Reddy, Burlington (CA)

(21) Appl. No.: 16/175,399

(22) Filed: Oct. 30, 2018

Related U.S. Application Data
(60) Provisional application No. 62/579,240, filed on Oct. 31, 2017.

(51) Int. Cl. A61B 5/08; A61B 5/087; G01N 33/497; G01N 33/00

(52) U.S. Cl. CPC A61B 5/082; A61B 5/087; G01N 2033/4975; G01N 33/0047; G01N 33/497

(57) ABSTRACT

This disclosure relates to portable devices and methods for detecting and identifying one or more compounds in the breath of a subject. In some embodiments, the devices and methods described herein detect and identify one or more compounds in real time.

This application claims priority under 35 U.S.C. § 119 (e) from U.S. Provisional Application Ser. No. 62/579,240, filed Oct. 31, 2017 which is hereby incorporated by reference in its entirety.

### TECHNICAL FIELD

This disclosure relates to portable devices and methods for detecting and identifying one or more compounds in the breath of a subject. In some embodiments, the devices and methods described herein detect and identify one or more compounds in real time.

### BACKGROUND

Substances in breath often provide evidence of ingestion of various materials in a subject, such as, for example, chemical compounds, including pharmaceutical compounds. Examples of compounds that can be detected in breath include, but are not limited to, apremilast, lenalidomide, sofosbuvir and velprasti, ledipasvir and sofosbuvir, cannabis, sofosbuvir, an opiate, hydromorphone, vitamin A, vitamin B, vitamin C, vitamin D, vitamin E, omega 3, omega 6 or omega 9, food additives, components of St. John's wart or peppermint oil, corresponding metabolic degradation products, etc.

An important application of compound detection in the breath of subjects is compliance with pharmaceutical prescriptions. Frequently, patients due to memory loss or simple forgetfulness, fail to ingest prescribed medication in a timely fashion or at all, which can lead to serious medical issues. Furthermore, health care professionals, who treat such patients are not aware of the lack of compliance, which may prevent proper remedial action. Currently, substances can be detected in breath by a number of methods (see, e.g., Mifsud et al, U.S. Pat. No. 5,801,297; Gordon et al., U.S. Pat. No. 9,709,582; Sood et al., International Publication No. WO 2012/1221283; and Laurenco et al., Metabolites 2014, 4, 465-498) but none measure patient compliance directly in real time. Pre-existing methods require burdensome sample collection and subsequent analysis with delay in reporting of results.

Accordingly, there exists a need for automated portable devices and methods for directly detecting compounds, in the breath of subjects which provide analytical results in real time with concurrent reporting to remote users, such, for example, health care professionals. Such devices and methods would be of significant value in measuring patient compliance with pharmaceutical regimens, thus ameliorating medical issues associated with the failure of subjects to ingest prescribed pharmaceuticals in a timely fashion.

### SUMMARY

The embodiments disclosed herein satisfies these and other needs by providing portable devices and methods for detecting and identifying one or more compounds in breath. In other aspects, coatings for tablets, novel tablets and novel sensors are provided.

In one aspect, a portable device for detecting and identifying one or more compounds in the breath of a subject is provided. The device includes a mouth piece connected to a housing, a sensor module disposed in the housing, which collects data that detects and identifies the one or more compounds in the breath of the subject, a communication apparatus disposed in the housing connected to the sensor module, which can transmit the data collected by the sensor module to an external processing apparatus and a battery disposed in the housing connected to the sensor module and the communication apparatus. In some embodiments, a processing apparatus is electrically or wirelessly connected to the communication apparatus, which analyzes data transmitted by the communication apparatus to detect and identify the one or more compounds in the breath of the subject. In still other embodiments, a dispenser is attached to the housing.

In another aspect, a method for detecting and identifying one or more compounds in the breath of a subject is provided. The method includes transmitting the breath of a subject collected in a mouthpiece to a housing, where the housing includes a sensor module, a communication apparatus and a battery, collecting data about the identity of the compound with the sensor module, transmitting the data with the communication apparatus to a processing apparatus and analyzing the communicated data with the processing apparatus to detect and identify the one or more compounds in the breath of the subject.

In still another aspect, a coating for a tablet is provided. The coating includes a nanoparticle, one or more marking compounds embedded in the nanoparticle and a polymer matrix.

In still another aspect, a capsule is provided. The capsule includes one or more compounds mixed with a composition comprising a nanoparticle, one or marking compounds embedder in the nanoparticle and a polymer matrix.

In still another aspect, a tablet is provided. The tablet includes one or more compounds in tablet form and a coating covering the tablet which includes a nanoparticle, one or marking compounds embedded in the nanoparticle and a polymer matrix.

In still another aspect, a tablet is provided. The tablet includes one or more compounds in tablet form and a coating covering the tablet which includes a functionalized inorganic metallic oxide nanoparticle and a polymer matrix.

In still another aspect, a sensor is provided. The sensor includes a counter electrode, a working electrode which includes multi-walled carbon nanotubes that are attached to one or more biological molecules, a reference electrode and a support on which the electrodes are disposed.

In still another aspect, a sensor is provided. The sensor includes a counter electrode, a working electrode which includes multi-walled carbon nanotubes, a reference electrode and a support on which the electrodes are disposed.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates an example of a portable device which identifies and detects one or more compounds in the breath of a subject.

FIG. 2 illustrates a process of identifying and detecting one or more compounds in the breath of a subject.

FIG. 3 illustrates a cross sectional view of a portable device which identifies and detects one or more compounds in the breath of a subject.

FIG. 4 illustrates a sensor module of a portable device which identifies and detects one or more compounds in the breath of a subject.

FIG. 5 illustrates an example of a sensor which may be used in the sensor module of a portable device which identifies and detects one or more compounds in the breath of a subject.

FIG. 6 illustrates a sensor which includes an immobilized biomolecule, which may be used in the sensor module of a portable device which identifies and detects one or more compounds in the breath of a subject.

FIG. 7 illustrates a neural network which may be used in a processing apparatus to analyze data provide by the sensor module.

FIG. 8 illustrates an example of a coated tablet.

FIG. 9 illustrates detection of vanillin embedded in chitosan nanoparticles at different concentrations ranging from 10 ppm to 100 ppm.

### DETAILED DESCRIPTION

Disclosed herein are portable devices and methods for detecting and identifying compounds in the breath of a subject. Also disclosed herein are coatings for tablets, novel tablets and novel sensors.

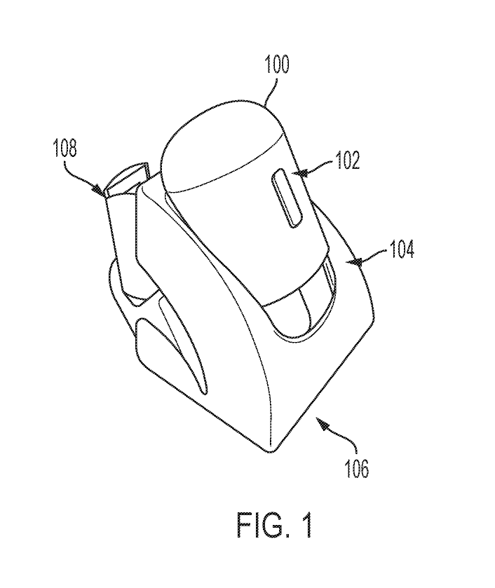

Referring to FIG. 1, illustrated is an example of a portable device 100, which may be used to detect and identify one or more compounds in the breath of a subject. The example depicted in FIG. 1 contains housing 102 which includes an attached mouthpiece (not shown), a sensor module (not shown), a communication apparatus (not shown), a charger 104, a dispenser 108 and stand 106. As is obvious to those of skill in the art, many other designs and configurations of such a portable device are possible and the above illustration is in no way limiting.

Briefly, a portable device, such as the one illustrated in FIG. 1 collects data about the presence and the identity of one or more compounds in the breath of a subject with a sensor module disposed in the housing. The sensor module converts collected data to a signal (e.g., optical, thermal, electrical, etc.), and transmits the data via a communication apparatus to a processing apparatus which analyzes the data to provide information about the presence and identity of one or more compounds in the breath of the subject. The processing apparatus may include, for example, a neural network, which processes the received signals to provide information about the presence and identity of the one or more compounds in the breath of a subject and which may transmit the presence and identity of the one or more compounds to a display.

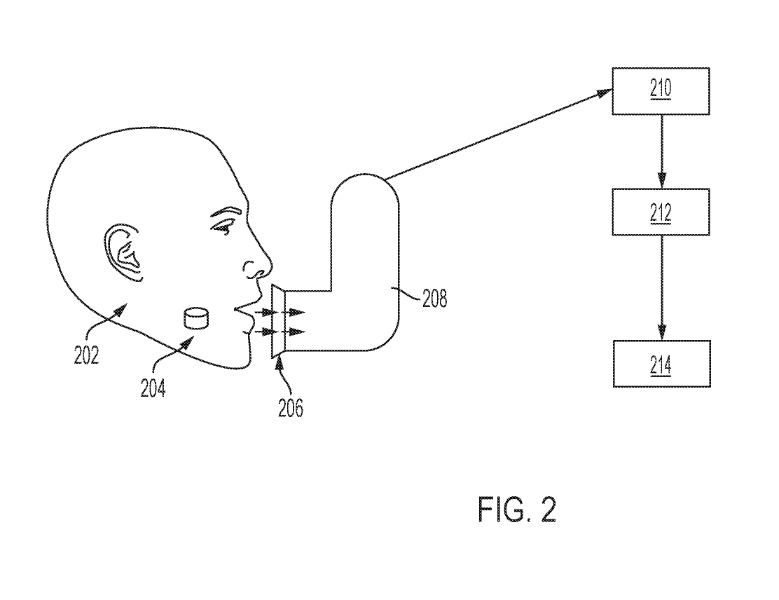

The process described above is illustrated in detail in FIG. 2. The subject 202 has ingested, for example, a tablet or capsule 204 including one or more compounds and breathes into housing 208, via mouthpiece 206. The breath of the subject enters the housing 208, which includes a battery (not shown) and a sensor module (not shown) and a communication apparatus (not shown), which transmits the data (e.g., by wireless or electrical means) collected by the sensor module, for example, to the cloud 210. The data may be stored in the cloud 210 and may be accessed by processing apparatus 212, which detects and identifies the compound in the breath of the subject and communicates the above result via a display to a user. The communication apparatus may directly transmit data to the processing apparatus 212, thus bypassing cloud 210. Ideally, the process illustrated in FIG. 2 takes place in real time (i.e., at substantially at the same time the event is occurring, with any delay being less than, for example, one minute). However, data may be stored in the cloud before being processed by the processing apparatus and displayed to a user.

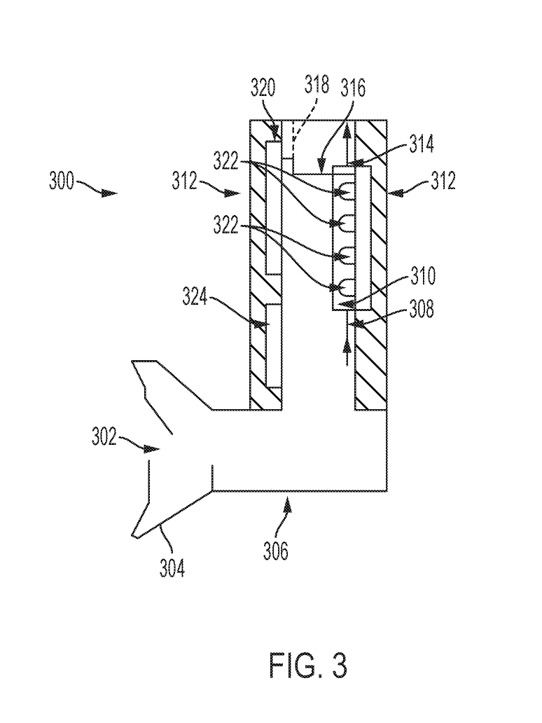

A portable device 300 is illustrated in a sectional view in FIG. 3. The device 300 has a mouthpiece 304 (see, e.g., Gump et al., U.S. Pat. No. 4,656,008 or the mouthpiece of any commercial breathalyzer for examples of mouthpieces which can be used in portable devices described herein) through which a subject provides a breath sample 302, which is connected to housing 306. Generally, housing 306 may be constructed from any material compatible with the described processes and may be substantially solid or hollow. In some embodiments, housing 306 is fabricated from plastic and includes casing 312 which is constructed of molded or printed plastic and serves to optimally position the various s disposed in housing 306. Housing 306 includes sensor module 310, which in this embodiment, has a vapor inlet 308, sensor array 320 and vapor outlet 314 and is attached to casing 312. Sensor module 310 is typically made of plastic and includes a desiccant (e.g., silica gel), and graphite or painted electrodes (i.e., working, counter and reference electrodes) and includes sensors 322. The electrodes are connected to a main circuit (not shown) with gold wire. Wire 316 connects communication apparatus 320 attached to casing 312 to sensor module 320. Wire 314 may be used to provide instructions from communication apparatus 320 or power from battery 324 to sensor module 320. Wire 318 allows communication apparatus to transmit data either wirelessly or electronically to a processing apparatus which is external to portable device 300.

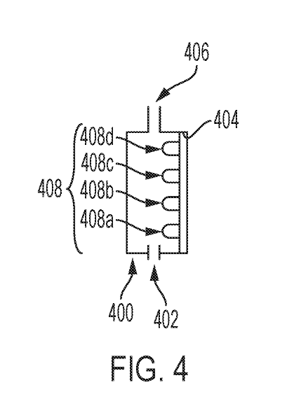

FIG. 4 illustrates sensor module 400 in greater detail. Inlet 402 is a tube which allows breath of a subject to enter the sensor module 400. Ideally, sensor module 400 will be positioned in the housing to receive the breath of the subject. Sensor module 400 includes an array of sensors 408A-D, which are independently capable of detecting the presence one or more compounds in the breath of a subject and are attached to base 404, which is typically molded plastic. In some embodiments, sensors 408A-D are selected to detect the presence of one or more compounds in the breath of the subject. After contact with sensors 408A-D, the breath of the subject may exit sensor module 400 through outlet 406. In certain embodiments, sensor module 400 is replaceable, which allows the portable device to be rapidly adapted to recognizing the presence and identity of many different types of compounds. It should be noted that the number of sensors can vary and is not limited by FIG. 4.

Sensors 408A-D, for example, may include metal oxides, electrically conducting inorganic or organic materials (e.g., zeolites, organometallic compounds, porous organic polymers, porous molecular solids) or biological materials such as enzymes, antibodies, nucleic acids, etc. to recognize the presence and identity of compounds in the breath of the subject. In general, the sensors described herein convert detection of the presence of one or more compounds in the breath of the subject to a signal, (e.g., electrical, optical, thermal, etc.) which is transmitted to a processing apparatus for analysis, identification and quantification.

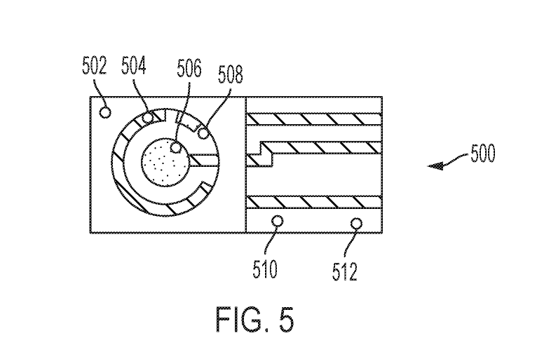

A simple electrochemical sensor for identifying and detecting one or more compounds in breath, for example, ascorbic acid, in the breath of a subject is illustrated in FIG. 5. Sample 502 encounters electrochemical sensor which includes counter electrode 504, working electrode 506, and reference electrode 508. The counter electrode 504 is, for example, made of carbon paint, while the reference electrode 508 is made for example, silver paint. Counter and reference electrodes also may be made of boron doped diamond, Ag or Pt. Electrodes which may be used in the sensors described herein include, but are not limited to, multiwalled carbon nanotubes (MWCNT)/Ag nanohybrids/Au, Ag nanoparticles/DNA/glassy carbon electrodes (GCE), nano-CuO/Ni/Pt, PtPdFe₃O₄ nanoparticles/GCE, Co₃O₄ nanoparticles/GCE, Cu₂O/Ni/Au, AgMnO₂-MWCNTs/GCE, etc. Application of voltage to working electrode 506, which includes carbon paint and multi-wall carbon nanotubes leads to recognition of the presence and identity of one or more compounds in the breath of the subject.

Multiwall carbon nanotubes, are an allotrope of carbon, having cylindrical structure and diameters, which range from less than 1 nm to about 100 nm in length. Multi-walled carbon nanotubes have many potentially applications in wide variety of industries due to many extraordinary properties coupled with nanometer-scale size and are accordingly well know and readily available. In some embodiments, multi-wall carbon nanotubes are used in the working electrodes

Other materials which may be used in working electrodes include but are not limited to superoxide dismutase, hemin, Zn-superoxide dismutase, superoxide dismutase-cysteine, xanthine oxidase, hypoxanthine, cytochrome C, cysteine, gold, sodium dodecyl sulfate, nanotube composites, silver nanoparticles, pyrolytic graphite, indium doped tin oxide, glassy carbon, carbon fiber, etc. In the electrochemical sensor illustrated in FIG. 5, the electrodes are arrayed, for example, on a glass epoxy chip 512 with an intervening insulating layer 510. Many other supports for disposing the electrodes are known to those of skill in the art and are entirely conventional. When ascorbic acid contacts working electrode 506 it is reduced, as is well known in the art, to dehydroascorbic acid as show below

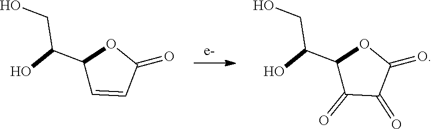

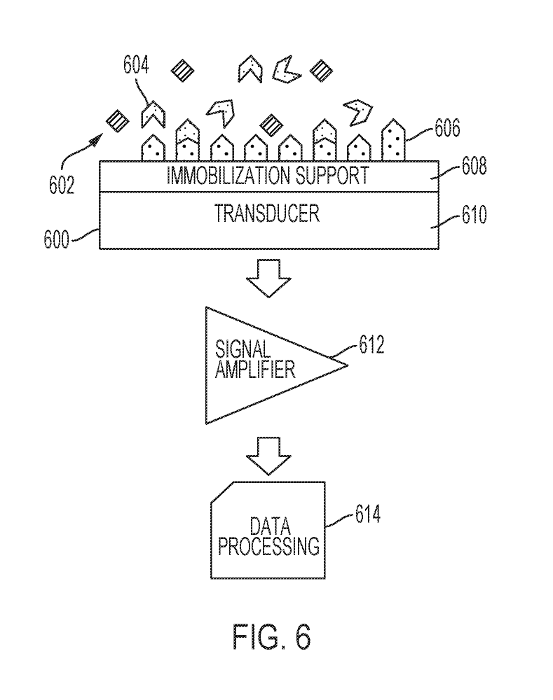

An exemplary sensor based on biological materials for use in a sensor module as described above is illustrated in FIG. 6. Sensor 600 includes biological molecules 606 immobilized on support 608. Biological receptors 606 include, for example, enzymes, cells, protein receptors, antibodies, nucleic acids, etc. Shown in FIG. 6 are constituents 602 which are present in the sample but do not bind to immobilized biological molecule 606 on support 608. Also shown in FIG. 6 is compound 604 which specifically bind to the biological molecule 606. Upon binding of compound 604 to immobilized biological molecule 606, a signal (e.g., electrical, optical or thermal) is generated which is communicated from transducer 610 to communication apparatus (not shown) and then to processing apparatus 614, via signal amplifier 612, where the data is processed to confirm the presence and identity of the compound. Signal amplifier 612 reduces instability and noise and may be purchased from commercial sources (see e.g., NICO2000 Ltd, 62 Pebworth Road, Harrow, Middlesex, HA1 3UE, U.K.; Smart Shape Enterprise (1240 Rockwell Ave, Suite 3A, Cleveland Ohio 44114). The signal may be directly communicated to the processing apparatus without the intermediacy of a signal amplifier, which is an optional.

Referring again to FIG. 5, a sensor based on biological materials can be constructed by covalently attaching a biological molecule to the multi-walled carbon nanotube component of the working electrode 506. In some embodiments, the biological molecule is an enzyme. In other embodiments, the biological molecule is an oxidase. In still other embodiments, the biological molecule is laccase. Laccase, a copper containing oxidase is well known in the art for oxidizing phenols (i.e., marker compounds) to quinones, as illustrated below.

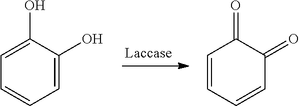 [SMILES: Oc1ccccc1O ; O=C1C=CC=CC1=O]

Laccase, after oxidizing phenols, transfers charge to the multi-walled carbons nanotube, thus leading to an electric signal. The sensitivity of the laccase system is very high with the ability to detect some phenols at a sensitivity as low as 1 ppb. For example, laccase can be attached to an electrode by incubation with 1-Ethyl-3-3(-dimethylamino propyl) carbodiimide, N-(2-hydroxyethyl) succinimide for two hours in phosphate buffer. The immobilized laccase was effective in oxidizing vanillin and other marker compounds, such as for example, catechol and guiacol in solution as demonstrated by cyclic voltammetry.

An exemplary communication apparatus can be purchased from commercial sources (e.g., Qualcomm® Snapdragon™ SDM845 X20 LTE modem from Qualcomm, Inc. San Diego, Calif.) and is entirely conventional. Many such communication apparati are known in the art and can be used in the portable devices described herein.

An exemplary battery is a lithium ion battery, which are conventional and available from many commercial sources (e.g., Panasonic DMW-BCM14 battery). Many batteries are known in the art and may be used in the portable devices described herein.

The processing apparatus will typically be a conventional general-purpose computer which includes a display device and a communication interface which allows reception and transmittal of information from other devices and systems via any communication interface. The processing module will typically detect and identify the one or more compounds in the breath of the subject by processing the data received from the sensor module with results sent to the display device. Any general purpose computer known in the art which has sufficient processing power to analyze data provided by the sensor module may be used in conjunction with the portable devices described herein.

In some embodiments, data from sensors in the sensor module is analyzed using pattern and recognition systems such as, for example, artificial neural networks, which include, for example, multi-layer perception, generalized regression neural network, fuzzy inference systems, etc. and statistical methods such as principal component analysis, partial least squares, multiples linear regression, etc. Artificial neural networks are data processing architectures that use interconnected nodes (i.e., neurons) to map complex input patterns with a complex output pattern. Importantly, neural networks can learn from using various input-output training sets.

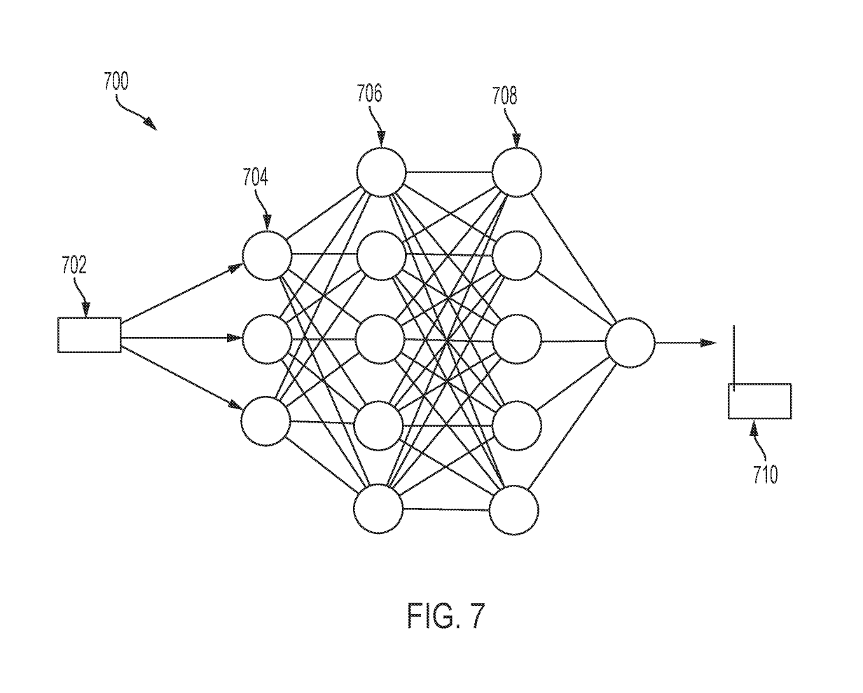

Referring now to FIG. 7, an exemplary artificial neural network 700 which can process data received from the sensor module 702 is illustrated. In general, the neural network uses three different layers of neurons. The first layer is input layer 704, which receives data from sensor module 702, the second layer in hidden layer 706 while the third layer is output layer 708, which provides the result of the analysis at 710. Note that each neuron in hidden layer 706 is connected to each neuro in input layer 704 and each neuron in output layer 708. In the exemplified neural network, hidden layer 706 processes data received from input layer 704 and provides the result to output layer 708. Although only one hidden layer is illustrated in FIG. 7, any number of hidden layers may be used, with the number of neurons limited only by processing power and memory of the general purpose computer. The inputs to the input neurons are inputs from the sensors in the sensor module. If, for example, seven sensors are in the sensor module, then the input layer will have seven neurons. In general, the number of output neurons corresponds to the number of compounds that the sensor module is trained to detect and identify. The number of hidden neurons may vary considerably. In some embodiments, the number of hidden neurons are between about 4 to about 10.

The portable device may include a dispenser for dispensing tablets of compounds, which are typically medically prescribed compounds (e.g., approved pharmaceuticals). The dispenser may be a cartridge or any convenient design which stores and dispenses tablets of compounds whose ingestion by the subject will be monitored by the portable device. Accordingly, a dispenser may be attached to the housing of the portable device described herein for the convenience of the subject. In some embodiments, compounds which are loaded into the dispenser are covered with a coating which included marker compounds which may be detected in the breath of a subject.

In some embodiments, the one or more compounds which are detected and identified using the portable devices are detected and identified directly. For example, if a certain pharmaceutical (e.g., morphine) is ingested by a subject it may be detected and identified as morphine by the portable device. Pharmaceutical compounds which may be directly identified and detected include, but are not limited to, apremilast, lenalidomide, sofosbuvir and velprasti, ledipasvir and sofosbuvir, cannabis, sofosbuvir, an opiate, hydromorphone, Jardiance, Januvia, atenol, lisinopril, amylodipine besylate, Cozaar, Tekturan, Revlamid, Ibrance, Imbruvica, Xtandi, Xytiga, Venclexat, Xeloda, Solvadi, Harvoni, Epclusa, atrovastatin, pravastatin, rosivastatin, pitvastatin, mycophenolate mofetil, folic acid, tetrahydrocannabinol, cyclosporine, Tacrolimus Januvia, Janumet, Eliquis, Xarelto or Sirolimus. Other compounds which may be identified and detected include, but are not limited to, vitamin A, vitamin B, vitamin C, vitamin D, vitamin E, omega 3, omega 6 or omega 9.

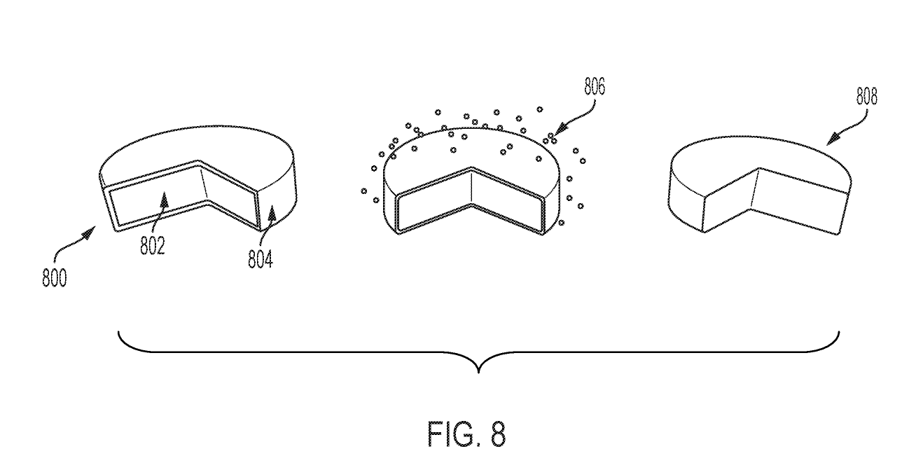

In other embodiments, however detection and identification of pharmaceutical compounds may involve the detection and identification of a marker compound, which has been used, for example, to coat an oral dosage form of the pharmaceutical compound. FIG. 8 illustrates the above concept. A tablet 800 includes one or more compounds 802 which is covered by coating 804. Coating 804 includes a nanoparticle with one or more embedded marker molecules which are disposed in a polymer matrix. After ingestion, the coating dissolves providing core tablet 808 comprised of the pharmaceutical compound and releasing the marker molecule 806 which is then detected and identified in the breath of the subject by the portable devices described herein. In general, the coating should dissolve in saliva so that the marker compound may be detected in the breath of the subject immediately after ingestion of the oral dosage.

In some embodiments, a coating for a tablet is provided. The coating includes a nanoparticle, a marker compound embedded in the nanoparticle and a polymer matrix. In other embodiments, a tablet is provided. The tablet includes one or more compounds in tablet form and a coating covering the tablet including a nanoparticle, one or more marking compounds embedded in the nanoparticle and a polymer matrix. In still other embodiments, a tablet is provided. The tablet includes one or more compounds in tablet form and a coating covering the tablet which includes a functionalized inorganic metallic oxide nanoparticle and a polymer matrix.

In some embodiments, the pharmaceutical compound which forms the core of the coated tablet is apremilast, lenalidomide, sofosbuvir and velprasti, ledipasvir and sofosbuvir, cannabis, sofosbuvir, an opiate, hydromorphone, Jardiance, Januvia, atenol, lisinopril, amylodipine besylate, Cozaar, Tekturan, Revlamid, Ibrance, Imbruvica, Xtandi, Xytiga, Venclexat, Xeloda, Solvadi, Harvoni, Epclusa, atrovastatin, pravastatin, rosivastatin, pitvastatin, Mycophenolate mofetil, folic acid, tetrahydrocannabinol, cyclosporine, Tacrolimus, Januvia. Janumet, Eliquis, Xarelto or Sirolimus.

In some embodiments, the nanoparticle is chitosan and a polymer, polyvinyl alcohol nanoparticles or polyvinylpyrolidine nanoparticles, which may be made by methods well known in the art. In other embodiments, the polymer used with chitosan is tripolyphosphate, HPMC, HPC, PVP, ethyl cellulose, PEG, cellulose acetate phthalate and derivatives thereof, bioadhesive coatings such as, for example, poly(butadiene-maleic anhydride-co-L-DOPA) (PBMAD), etc.

In some embodiments, the marker compound is a phenol. In other embodiments, the phenol is catechol, vanillin or guaicol. In still other embodiments, the polymer matrix used to form the coating is Dextran, esters of alcutric acid, cellulose acetate phthalate, poly(methacrylic acid-co-methyl methacrylate, cellulose acetate trimellitate, poly(vinyl acetate phthalate or hydroxypropyl methyl cellulose phthalate, etc.

In some embodiments, the marker compound is a functionalized inorganic metallic oxide nanoparticle. In other embodiments, the functionalized inorganic metallic oxide is ZnO nanoparticles functionalized with citric acid. ZnO nanoparticles can be made via conventional methods (Meulenkamp, J. Phy. Chem B 1998, 5566; Vaseem et al., Chapter 4 in Metal Oxide Nanostructures and their Applications, edited by Ahmad Umar and Yoon Bong Hahn, Volume 5). Ultrasonication of the ZnO particles with citric acid provides ZnO nanoparticles functionalized with citric acid, which is detected by the sensors in the sensor module.

In some embodiments, the thickness of the coating is between about 0.1 microns and about 100 microns. In other embodiments, the thickness of the coating is between about 0.2 microns and about 25 microns thick. In still other embodiments, the thickness of the coating is between about 0.3 microns and about 10 microns thick. In still other embodiments, the thickness of the coating is about 0.5 microns.

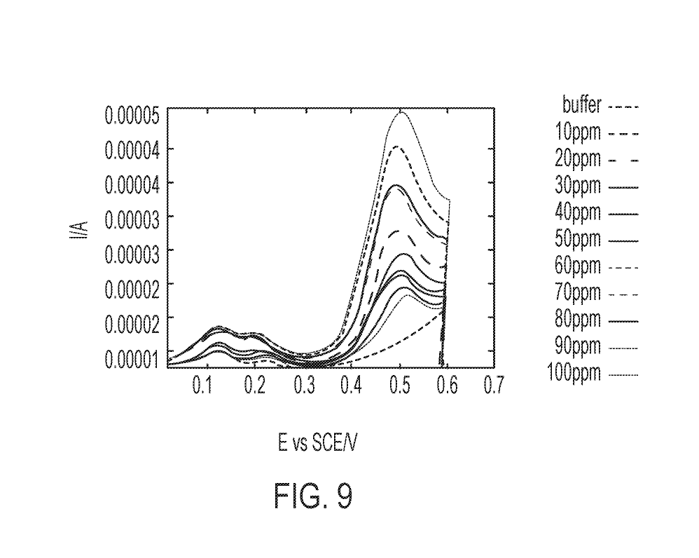

Those of skill in the art will appreciate that combinations of nanoparticles with different marker compounds at varying concentrations can be used to create a vast library of unique coatings. For example, coatings which includes the phenol vanillin embedded in chitosan nanoparticles at different concentrations ranging from 10 ppm to 100 ppm can be readily distinguished as illustrated in FIG. 9. Coatings which include chitosan nanoparticles complexed with 100 ppm each of vanillin, catechol and guiacol can be readily distinguished from coatings which include the above phenols at any number different concentrations because of the great sensitivity of the laccase assay. The above examples represent only a small fraction of unique coatings which can be made. In some embodiments, the concentration of vanillin which can be identified and detected is as low as 1 ppm, 0.025 ppm or 1 ppb.

The number of different coatings increases dramatically with the number of different marker compounds which are used and the number of different concentrations of marker compounds which can be detected. The marker compounds and concentration of marker compounds can be varied systematically to create and almost infinite number of different coatings which can be distinguished Examples of different coatings that can be detected, include but are not limited to the following: 100 ppm load of chitosan nanoparticle complexed with vanillan, catechol and guiacol; 50 ppm load of chitosan nanoparticle complexed with catechol and guiacol; 200 ppm load of chitosan nanoparticle complexed with vanillan and guiacol; 150 ppm load of chitosan nanoparticle complexed with vanillan and catechol; and 50 ppm load chitosan complexed with vanillan, 100 ppm load chitosan complexed with catechol and 150 ppm load guiacol complexed with catechol.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A portable device for detecting and identifying one or more compounds in the breath of a subject comprising:
a mouth piece connected to a housing;
a sensor module disposed in the housing which collects data which detects and identifies the one or more compounds;
a communication apparatus disposed in the housing connected to the sensor module which electrically transmits the data collected by sensor module; and
a battery disposed in the housing connected to the communication apparatus and the sensor module.

2. The portable device of claim 1, wherein a processing apparatus which processes the data transmitted by the communication apparatus to detect and identify the compound is electronically or wirelessly connected to the communication apparatus.

3. The portable device of claim 2, wherein the processing apparatus transmits the identity of the compound detected in the sample from the processing apparatus to a display.

4. The device of claim 1, further comprising a tablet dispenser attached to the housing.

5. The device of claim 1, further comprising an amplifier connected to the sensor module which amplifies data collected by the sensor module in the housing.

6. The device of claim 1, wherein the one or more compounds are detected in real time.

7. The device of claim 1, wherein the one or more compounds is apremilast, lenalidomide, sofosbuvir and velprasti, ledipasvir and sofosbuvir, cannabis, sofosbuvir, an opiate, hydromorphone, Jardiance, Januvia, atenol, lisinopril, amylodipine besylate, Cozaar, Tekturan, Revlamid, Ibrance, Imbruvica, Xtandi, Xytiga, Venclexat, Xeloda, Solvadi, Harvoni, Epclusa, atrovastatin, pravastatin, rosivastatin, pitvastatin, Mycophenolate mofetil, folic acid, tetrahydrocannabinol, cyclosporine, Tacrolimus or Sirolimus.

8. The device of claim 7, wherein the one or more compounds is coated with one or more nanoparticles which include one or more marker compounds.

9. The device of claim 8, wherein the compound identified and detected is a marker compound.

10. The device of claim 9, wherein the marker compound is a phenol.

11. The device of claim 10, wherein the phenol is catechol, vanillin or guaicol.

12. The device of claim 9, wherein the marker compound is a functionalized inorganic metallic oxide nanoparticle.

13. The device of claim 4, wherein the tablet dispenser dispenses tablets which includes the one or compounds detected and identified by the device.

14. A sensor comprising:
a counter electrode;
a working electrode comprising multi-walled carbon nanotubes which includes one or more immobilized biological molecules;
a reference electrode; and
a support on which the electrodes are disposed.

15. A method for detecting and identifying one or more compounds in the breath of a subject comprising:
transmitting the breath of a subject collected in a mouthpiece connected to a housing, wherein the housing includes a sensor module, a communication apparatus and a battery;
collecting data about the presence and identity of the one or more compounds with the sensor module;
communicating data via the communication apparatus to a processing apparatus; and;
processing the communicated data to detect and identify the one or more compounds.
